(19) United States
(12) Patent Application Publication
(10) Pub. No.: US 2014/0349303 A1
(43) Pub. Date: Nov. 27, 2014

(54) System and Method for Producing and Reading DNA Barcodes

(71) Applicant: Sunpower Technologies LLC, San Marcos, CA (US)

(72) Inventor: Travis Jennings, San Diego, CA (US)

(73) Assignee: Sunpower Technologies LLC, San Marcos, CA (US)

(21) Appl. No.: 13/901,710

(22) Filed: May 24, 2013

(51) Int. Cl. C12Q 1/68

(52) U.S. Cl. CPC C12Q 1/6895
USPC 435/6.19; 435/287.2

(57) ABSTRACT

The present disclosure relates to biological material identification systems and methods. DNA oligomers may be used to encode for specific characteristics of biological materials. Encoding may be done by depositing suitable amounts of DNA oligomers onto a portion of a biological material. To identify the biological materials, the encoded portions of the biological materials may be extracted and immersed in buffer solutions. Then, lateral flow tests may be used to decode the DNA for interpretation, creating a readable barcode that may be compared with a database to determine if the biological material may be approved.

### BACKGROUND

1. Field of the Disclosure

The present disclosure relates generally to biological material ID systems, and more particularly DNA barcodes.

2. Background Information

In many cases the movement and distribution of biological materials, such as plants, crops, and seeds, have to be tracked and controlled for various reasons.

In research facilities and in industries, such as medical and healthcare industries, biological materials are used to derive substances that may be applied in therapeutic treatment of illnesses, but illegal, toxic substances may also be derived from these biological materials, raising the need to track and control the movement of the biological materials.

There are few methods for identifying legal biological material products from illegal varieties; however, there are certain methods that may modify these biological material products or their production, which may be considered neither convenient nor accurate, and may represent a high cost for several regulation entities. For example, the use of genetic engineering may innately modify the plant in a very fundamental form.

There is therefore a need to be able to distinguish authorized biological materials from common, illegal, or toxic varieties of biological materials; a new method may be applied to perform the identification of legal and illegal biological materials with more accuracy and lower cost.

### SUMMARY

Systems and methods for biological material identification are disclosed. DNA oligomers may be used to encode for specific characteristics of biological materials.

The process for barcode identification of biological materials may begin with a biological material preparation process, in which a suitable amount of synthetic encoding strands DNA in solution may be deposited onto at least a portion of the surface of the biological material that will later be identified, using suitable methods, including sprays, droppers, and gels, among others. Afterwards, the applied DNA oligomers solution (encoding strands) may be left to dry at ambient temperature or may by dried by other means. When the biological material needs to be identified, a sample from the region onto which the DNA oligomers were deposited may be extracted. Then, the extracted sample may be immersed in a suitable buffer solution. This buffer may simply be a diluent or running buffer or it may be a complex buffer, having specific components or properties required to make the strip perform properly.

After encoding, lateral flow tests may be used to decode the DNA oligomers for interpretation, creating a readable barcode that may be compared with a database to determine if the biological materials come from approved growth facilities, belong to a specific breed, or possess specific properties or characteristics.

The encoding may be done directly depositing a solution including one or more oligonucleotides onto the product, or by including a suitable substrate already encoded with oligonucleotides. Oligomers may be extracted from the product by soaking the sample part of the plant or encoded substrate in an appropriate volume of buffer solution. Before performing a lateral flow test, the sample may be filtered if necessary through either a common 0.22 μm syringe filter, or a filter integrated into the assay device.

Then, an appropriate amount of sample solution may be placed into a lateral flow test strip, designed in such a way that encoding strands of DNA, if present, may produce a visibly detectable barcoded readout along the detection membrane. To generate the visible barcode, visible markers may be used. Visible markers may include colloidal metals such as gold or silver, carbon, a visible or florescent dye, magnetic particles, enzymes, latex beads impregnated with visual or fluorescent dyes, or a combination of these which are conjugated to either an antibody or antigen to generate signal.

Suitable scanning devices, including a mobile computing device (e.g., a smartphone, cell phone, or tablet computer) and barcode scanners, may be used to scan the generated barcode. Then, the barcode may be interpreted by comparison with a secure database library, to decide if the biological material complies with a set of predefined requirements. In some embodiments, these requirements may include plant breed, content percentage of specific compounds and growth facility, among others.

In one embodiment, the disclosed method may be used to track or identify, or both, biological materials, such as genetically engineered plants or seeds.

In one embodiment, the present disclosure provides means to distinguish medical marijuana from common, illegal varieties of cannabis. This is achieved by producing and reading out DNA barcodes from specifically encoded marijuana plants.

In another embodiment, the disclosed methods may be utilized to track, identify, or both, other plants from which controlled substances may be derived, like opium poppy or coca plants.

In one embodiment, a lateral flow test strip comprises a sample pad configured to receive a sample solution; a conjugate pad comprising detection conjugates, wherein the detection conjugates are configured to bind to encoding strands included in the sample solution to create a complex of detection conjugates and sample solution, and wherein the detection conjugates carry visible markers; a detection membrane configured to receive the complex and migrate the complex along the detection membrane; test lines comprising immobilized capture strands on a first section of the detection membrane, wherein each capture strand is configured to attach to a predetermined encoding strand, and when an encoding strand binds to a capture strand, a visible bar code forms on the surface of the detection membrane as a result of the visible markers carried by the encoding strands; and control lines on a second section of the detection membrane to collect excess complex and indicate if a lateral flow test is complete by binding to excess encoding strands that do not bind to the test lines. The lateral flow test strip may further comprise an adsorbent pad configured to pull the complex off the detection membrane to keep the complex flowing on the detection membrane; and a backing card supporting the sample pad, the conjugate pad, the detection membrane, the test lines, the control lines, and the adsorbent pad.

In another embodiment, a method for marking and identifying a plant comprises: depositing a DNA oligomer solution including encoding strands onto a portion of the plant; drying the DNA oligomer solution on the plant; extracting a sample of the plant from the portion of the plant where the DNA oligomer solution was deposited; immersing the extracted sample in a buffer solution; depositing a portion of the buffer solution including the extracted sample and the encoding strands onto a lateral flow test strip; performing a lateral flow test using the lateral flow test strip, thereby generating a bar code on the lateral flow test strip as a result of the lateral flow test and a detection reaction performed by the lateral flow test strip; scanning the bar code using a scanner; and comparing the scanned bar code to stored bar codes in a library database to identify a characteristic about the plant.

Additional features and advantages of an embodiment will be set forth in the description which follows, and in part will be apparent from the description. The objectives and other advantages of the invention will be realized and attained by the structure particularly pointed out in the exemplary embodiments in the written description and claims hereof as well as the appended drawings.

It is to be understood that both the foregoing general description and the following detailed description are exemplary and explanatory and are intended to provide further explanation of the invention as claimed.

### BRIEF DESCRIPTION OF THE DRAWINGS

The present disclosure can be better understood by referring to the following figures. The components in the figures are not necessarily to scale, emphasis instead being placed upon illustrating the principles of the disclosure. In the figures, reference numerals designate corresponding parts throughout the different views.

FIG. 1 is a flowchart of a biological material preparation process, according to an embodiment.

FIG. 2 shows components of a lateral flow test, according to an embodiment.

FIG. 3 illustrates a detection reaction, according to an embodiment.

FIG. 4 shows an identification barcode, according to an embodiment.

FIG. 5 is a flowchart of a biological material identification process, according to an embodiment.

### DETAILED DESCRIPTION

The present disclosure is here described in detail with reference to embodiments illustrated in the drawings, which form a part here. Other embodiments may be used and/or other changes may be made without departing from the spirit or scope of the present disclosure. The illustrative embodiments described in the detailed description are not meant to be limiting of the subject matter presented here.

### DEFINITIONS

As used here, the following terms may have the following definitions:

“DNA oligomers” refers to short single-stranded sequences of deoxyribonucleic acid (DNA) formed by bounded molecules.

“Encoding strands” refers to DNA oligomer sequences used in lateral flow tests to generate barcodes.

“Barcode” refers to a pattern that allows the identification or verification of the type of a living being based on a DNA sequence.

“Biological material” refers to substances containing genetic information from organisms of the Plantae kingdom, such as plants and seeds, capable of reproducing themselves or being reproduced in a biological system.

### DESCRIPTION OF THE DRAWINGS

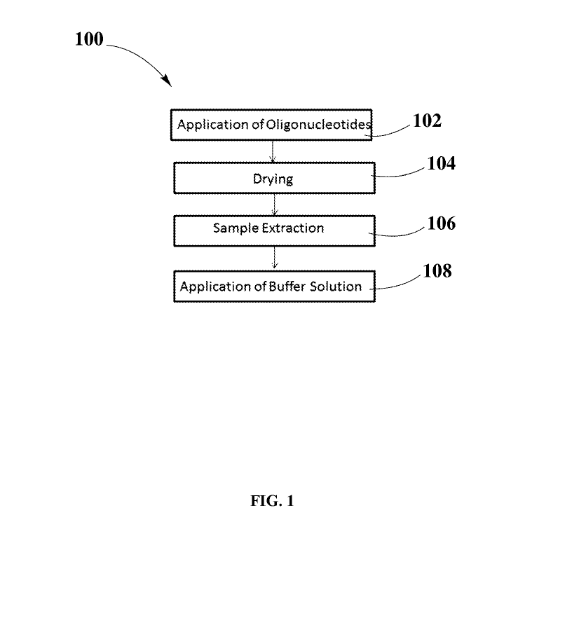

The process for a barcoded identification of a biological material may begin with biological material preparation process 100, shown in FIG. 1. Biological material preparation process 100 may start with application of oligonucleotides 102, in which a suitable amount of synthetic encoding strands in solution may be deposited on a biological material that may later be identified using suitable methods, including sprays, droppers, and gels, among others. Following application of oligonucleotides 102, the applied DNA oligomers solution (encoding strands) may be left to dry at ambient temperature or may by dried by other means during drying 104. When the biological material needs to be identified, a sample from the region onto which the DNA oligomers were deposited may be extracted, as part of sample extraction 106. Then, during application of buffer solution 108, the extracted sample may be immersed in a suitable solution. This buffer solution may simply be a diluent or running buffer or it may be a complex buffer, having specific components or properties required to make the strip perform properly. In one embodiment, about 0.1 to about 30 mL of phosphate buffered saline (PBS) may be used, depending on the sample size.

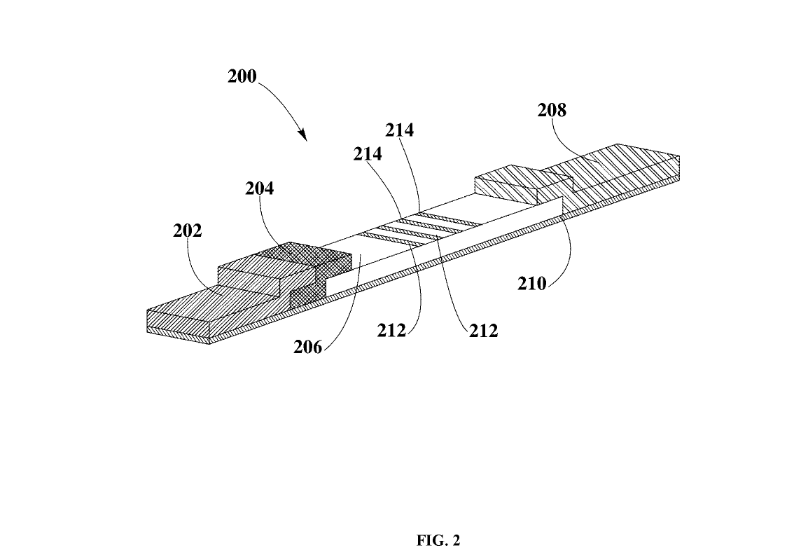

FIG. 2 shows a lateral flow test strip 200, which may include at least a sample pad 202, a conjugate pad 204, a detection membrane 206, an adsorbent pad 208, a backing card 210, test lines 212, and control lines 214.

Sample pad 202 may be made of cellulose, glass fiber, or other material where a fluid sample may be applied to lateral flow test strip 200 and, if necessary, material of sample pad 202 may modify the properties of the fluid in order to improve the results of the assay. This might be achieved by modifying pH, filtering out unwanted solid components, separating whole blood constituents, adsorbing out unwanted antibodies, or improving some other test specific variable.

Conjugate pad 204 may be made of non-absorbent materials such as fiberglass pad, polyester, rayon, or similar materials. Conjugate pad 204 is commonly made of synthetic materials, when using a gold conjugate, to ensure the efficient release of its contents. Conjugate pad 204 may include one or more detection conjugates.

The detection conjugates may be dried down and held in conjugate pad 204 until a liquid test sample is applied to sample pad 202. The liquid from the sample solution may move, by capillary action, into conjugate pad 204, in addition to re-hydrating the dried conjugate and allowing the mixing of the sample with the conjugate. At this point, the detection conjugates carrying visible markers may start to bind to encoding strands. In some embodiments, different detection conjugates may be present in conjugate pad 204, each one being capable to bind to different encoding strands. The complex of conjugate and sample solution may then move into detection membrane 206. Pre-treatment of the conjugate pad 204 may help to ensure the conjugate releases at the proper rate and enhances the stability of conjugate pad 204.

As visible markers, lateral flow test strips 200 may include colloidal metals such as gold or silver, carbon, a visible or florescent dye, magnetic particles, enzymes, latex beads impregnated with visual or fluorescent dyes, or a combination of these which are conjugated to either an antibody or antigen to generate signal.

Commonly, lateral flow test assays, may include gold, colored latex or another visually observable particle adsorbed with antibodies or antigens specific to the analyte being detected. When the strip is designed to be used with a reader, the conjugate may not be easily visible or interpreted to the naked eye. If the strip will be read visually, the detection particle must be large enough to be seen but not so large as to overwhelm the complementary DNA strand conjugated to the surface of the detection particle. These particles may have a size between about 1 and about 100 nm.

Detection membrane 206 may be a thin Mylar sheet coated with a layer of nitrocellulose (NC). NC binds proteins electrostatically through an interaction with the nitrate esters and the peptide bonds of the protein. Therefore, DNA binding to the nitrocelluose membrane may be accomplished by first conjugating the capture DNA strands to proteins (such as Bovine Serum Albumin, or BSA) and then patterning the nitrocellulose with the BSA-DNA conjugate. Detection membranes 206 binding capacity is ultimately determined by the available surface area. This surface area is determined by pore size, porosity (pore density), thickness, and unique physical characteristics of that particular polymer. These factors also affect capillary flow rate, which may also dramatically affect a lateral flow test's overall performance. If a strip flows too fast sensitivity may be compromised, and if the strip flows too slowly, specificity may be compromised.

As with many immunological based assays, blocking may be necessary to prevent nonspecific binding of sample and conjugate to the test lines and to limit background along detection membrane 206. Blocking is also used to control flow rates and stabilize test and control-line proteins. The blocking process involves immersion of the striped detection membrane 206 in an aqueous solution of proteins, surfactants, and/or polymers. Detection membrane 206 may then be removed, blotted, and dried.

The complex of detection conjugate and sample solution may move onto detection membrane 206 and continue to migrate towards the capture binding proteins immobilized in the test lines 212. The capture binding proteins may include antibodies or antigens specifically designed to capture encoding strands. When encoding strands bind to capture strands and conjugate detection strands, a visible signal may form in the surface of detection membrane 206. Then the excess complex may reach a second section, the control lines 214. Here, the excess conjugate may bind to antibodies or antigens will capture the visible markers to indicate if the test is successful.

Afterwards, the excess fluid may continue to move towards adsorbent pad 208 which, also called a wick or wicking pad, pulls fluid off of detection membrane 206 to allow the capillary flow, to keep the mixture flowing in the proper direction and at the proper rate. Most absorbent pads 208 are made from non-woven, cellulose fiber sheets. These pads may be manufactured in a variety of thicknesses and densities to suit the needs of the assay.

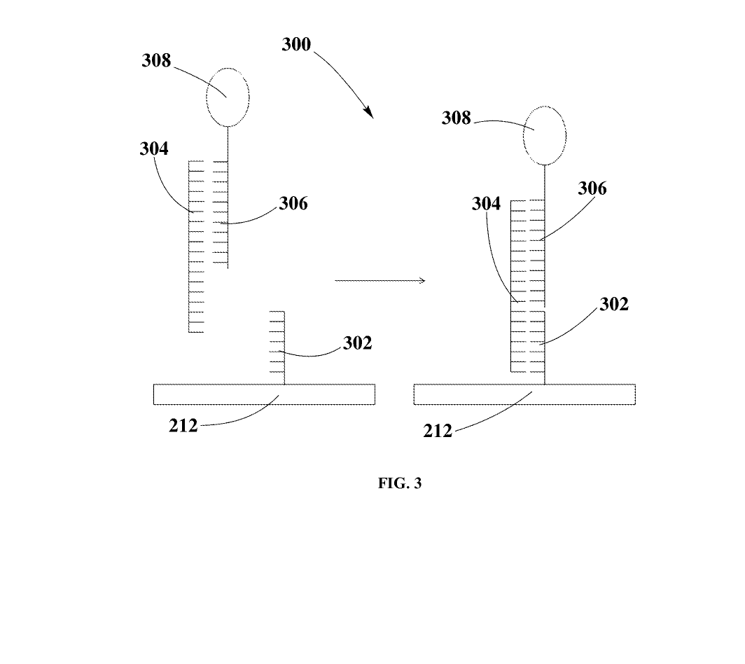

FIG. 3 illustrates detection reaction 300, which may be a hybridization reaction that occurs on detection membrane 206. At each of the test lines 212 that form a barcode, capture strands 302, specifically designed to attach to a certain type of encoding strand 304, may bind with encoding strands 304. Simultaneously, encoding strands 304 may bind with detection strands 306 included in the conjugate. Detection strands 306 may carry a visible marker 308. According to some embodiments, the visible marker 308 may be a metallic nanoparticle, such as gold or silver nanoparticles or color infused polystyrene nanoparticle. Other suitable, known in the art nanoparticles may be used. The nanoparticles used as visible markers 308 may have a diameter between about 1 and about 100 nm. Some nanoparticles may have a diameter between about 1 and about 30 nm.

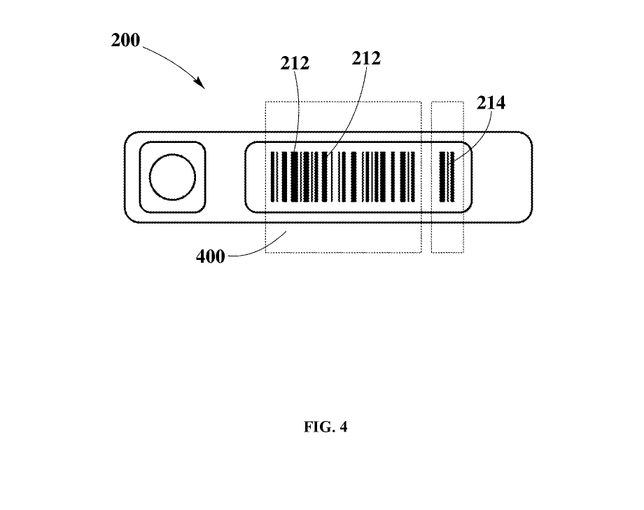

FIG. 4 shows a barcode 400 generated on the test lines 212 of lateral flow test strip 200 after a successful detection reaction 300. Note that also control lines 214 are visible.

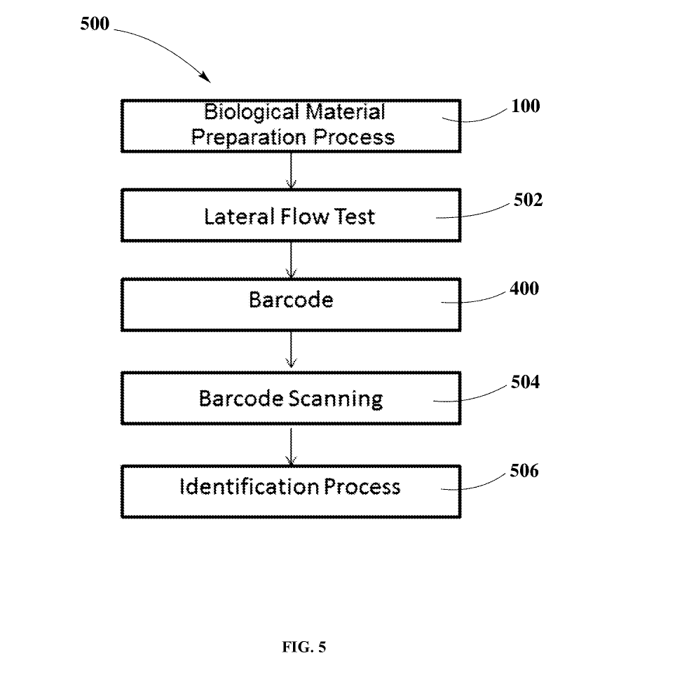

FIG. 5 is a flowchart of biological material identification process 500, which may start with biological material preparation process 100. Then a few drops of the buffer solution including encoding strands may be deposited on sample pad 202 of lateral flow test strip 200 to perform lateral flow test 502. After lateral flow test 502, barcode 400 may be generated as a result of detection reaction 300. Then, in barcode scanning 504, suitable scanning devices, including a mobile computing device (e.g., a smartphone, cell phone, or tablet computer) and barcode scanners, may be used to scan barcode 400. Following barcode scanning 504, barcode 400 may be interpreted by comparison with a secure database library, as part of identification process 506, to decide if the biological material complies with a set of predefined requirements. In some embodiments these requirements may include breed, content percentage of specific compounds and growth facility, among others.

### Examples

In example #1 a plant is identified following biological material identification process 500. After harvest the plant is sprayed with suitable DNA oligomers in solution and left to dry. Afterwards, the plant is packed and shipped. At a storage facility, the plant is identified, prior to being sold to a costumer. A sample from the plant is taken, including the DNA oligomers. The sample is submerged in a suitable buffer solution for a reasonable length of time, between about 1 and about 20 min. Then the mixture is deposited in a lateral flow test strip 200. As a result of the test, a barcode 400 may be generated. Barcode 400 is read using a smartphone and it is compared with a secure database of allowed results. The plant is positively identified and sold.

In example #2 a cannabis plant is identified following biological material identification process 500. After harvest the cannabis plant is sprayed with suitable DNA oligomers in solution and left to dry. Afterwards, the cannabis plant is packed and shipped. At a storage facility, the cannabis plant is identified, prior to being sold to a costumer. A sample from the cannabis plant is taken, including the DNA oligomers. The sample is submerged in a suitable buffer solution for a reasonable length of time, between about 1 and about 20 min. Then the mixture is deposited in a lateral flow test strip 200. As a result of the test a barcode 400 may be generated. Barcode 400 is read using a smartphone and it is compared with a secure database of allowed results. The cannabis plant is positively identified and sold.

In example #3 a coca plant is identified following biological material identification process 500. After harvest the coca plant is sprayed with suitable DNA oligomers in solution and left to dry. Afterwards, the coca plant is packed and shipped. At a storage facility, the coca plant is identified, prior to being sold to a costumer. A sample from the coca plant is taken, including the DNA oligomers. The sample is submerged in a suitable buffer solution for a reasonable length of time, between about 1 and about 20 min. Then the mixture is deposited in a lateral flow test strip 200. As a result of the test a barcode 400 may be generated. Barcode 400 is read using a smartphone and it is compared with a secure database of allowed results. The coca plant is positively identified and sold for the production of anesthetics.

In example #4 a cargo of opium poppy is identified following biological material identification process 500. After harvest the opium poppy is sprayed with suitable DNA oligomers in solution and left to dry. Afterwards, the opium poppy is packed and shipped. At a storage facility, the opium poppy is identified, prior to being sold to a costumer. A sample from the opium poppy is taken, including the DNA oligomers. The sample is submerged in a suitable buffer solution for a reasonable length of time, between about 1 and about 20 min. Then the mixture is deposited in a lateral flow test strip 200. As a result of the test a barcode 400 may be generated. Barcode 400 is read using a suitable barcode 400 reader and it is compared with a secure database of allowed results. The opium poppy is positively identified and sold for the production of analgesics and muscle relaxants.

In example #5 a cargo of genetically enhanced seeds is identified following biological material identification process 500. After harvest a sample from the seeds is sprayed with suitable DNA oligomers in solution and left to dry. Afterwards, the seeds are packed and shipped. At a storage facility, the seeds are identified, prior to being sold to a costumer. A sample from the seeds is taken, including the DNA oligomers. The sample is submerged in a suitable buffer solution for a reasonable length of time, between about 1 and about 20 min. Then the mixture is deposited in a lateral flow test strip 200. As a result of the test a barcode 400 may be generated. Barcode 400 is read using a suitable barcode 400 reader and it is compared with a secure database of allowed results. The seeds are positively identified and sold.

While various aspects and embodiments have been disclosed, other aspects and embodiments are contemplated. The various aspects and embodiments disclosed are for purposes of illustration and are not intended to be limiting, with the true scope and spirit being indicated by the following claims.

The embodiments described above are intended to be exemplary. One skilled in the art recognizes that numerous alternative components and embodiments that may be substituted for the particular examples described herein and still fall within the scope of the invention.

1. A lateral flow test strip comprising:
a sample pad configured to receive a sample solution;
a conjugate pad comprising detection conjugates, wherein the detection conjugates are configured to bind to encoding strands included in the sample solution to create a complex of detection conjugates and encoding strands and wherein the detection conjugates carry visible markers;
a detection membrane comprising a sample solution and configured to receive the complex and migrate the complex along the detection membrane;
test lines comprising immobilized capture strands on a first section of the detection membrane, wherein each capture strand is configured to attach to a predetermined encoding strand present in the sample solution in the detection membrane, and when an encoding strand binds to a capture strand, a visible bar code forms on the surface of the detection membrane as a result of the visible markers carried by the encoding strands; and
control lines on a second section of the detection membrane to collect excess complex and indicate if a lateral flow test is complete by binding to excess encoding strands that do not bind to the test lines.

2. The lateral flow test strip of claim 1, wherein the sample pad modifies the properties of the sample solution by modifying pH of the sample solution, filtering out unwanted solid components, separating whole blood constituents, absorbing out unwanted antibodies, or improving a test specific variable.

3. The lateral flow test strip of claim 1, wherein the sample pad comprises a non-woven cellulose fiber or glass fiber.

4. The lateral flow test strip of claim 1, wherein the visible markers comprise gold, silver, carbon, a fluorescent dye, magnetic particles, enzymes, latex beads impregnated with visual dyes, latex beads impregnated with fluorescent dyes, or a combination thereof.

5. The lateral flow test strip of claim 1, wherein the visible markers are metallic nanoparticles each having a diameter between 1 and 100 nm.

6. The lateral flow test strip of claim 1, wherein the detection membrane comprises a biaxially-oriented polyethylene terephthalate sheet coated with a layer of nitrocellulose.

7. The lateral flow test strip of claim 1, wherein the detection membrane is subject to a blocking process before receiving the complex.

8. The lateral flow test strip of claim 1, wherein the conjugate pad comprises fiberglass or polyester rayon.

9. The lateral flow test strip of claim 1, further comprising an adsorbent pad comprising a non-woven, cellulose fiber sheet.

10-20. (canceled)
